(19) United States
(12) Patent Application Publication
(10) Pub. No.: US 2026/0248745 A1
(43) Pub. Date: Aug. 27, 2026

(54) Thermogenic Composition for Cold Exposure

(71) Applicant: BioSentinel Inc, Martinez, CA (US)

(72) Inventor: Cihan Fuat Atkin, Martinez, CA (US)

(21) Appl. No.: 19/552,812

(22) Filed: Feb. 27, 2026

Related U.S. Application Data
(60) Provisional application No. 63/763,956, filed on Feb. 27, 2025.

(51) Int. Cl. A61K 31/137; A61K 9/00; A61K 9/08; A61K 31/165; A61K 31/185; A61K 31/198; A61K 31/205; A61K 31/46; A61K 31/522; A61K 31/714; A61K 33/00; A61K 33/06; A61K 36/67; A61K 36/82; A61K 36/906; A61K 36/9064

(52) U.S. Cl. CPC A61K 31/137; A61K 9/0019; A61K 9/08; A61K 31/165; A61K 31/185; A61K 31/198; A61K 31/205; A61K 31/46; A61K 31/522; A61K 31/714; A61K 33/00; A61K 33/06; A61K 36/67; A61K 36/82; A61K 36/906; A61K 36/9064

(57) ABSTRACT

A thermogenic composition prevents the adverse effects of cold exposure and/or mitigates hypothermia. Epinephrine, atropine sulfate, phenylephrine hydrochloride, L-carnitine, vitamin B12, magnesium sulfate, and saline solution are homogeneously mixed to form an injectable formulation for the thermogenic composition. Capsaicinoids, capsinoids, ginger extract, L-citrulline, grains of paradise extract, taurine, caffeine anhydrous, L-theanine, green tea extract, black pepper extract, potassium citrate, sodium citrate, and magnesium citrate anhydrous are heterogeneously mixed to form a capsulated formulation for the thermogenic composition.

### CROSS REFERENCE TO RELATED APPLICATIONS

The current application claims a priority to the U.S. provisional patent application serial number 63/763,956 filed on February 27, 2025.

### FIELD OF THE INVENTION

The present invention generally relates to thermogenic formulations designed to prevent and mitigate the effects of hypothermia by inducing controlled heat production and improving circulation in individuals exposed to extreme cold environments. More specifically, the formulations of the present invention are available in multiple delivery methods, including a sublingual strip [i.e., drug-based or over-the-counter (OTC) dietary supplement], an auto-injector (IM), an oral gel (i.e., drug-based), and OTC dietary supplements (i.e., for both adults and children).

### BACKGROUND OF THE INVENTION

Cold exposure and hypothermia pose significant risks in extreme environments, including military operations, outdoor survival scenarios, and space or deep-sea exploration. Existing methods such as thermal blankets, heat packs, and passive warming techniques are slow, ineffective in critical situations, and dependent on external heat sources.

Therefore, an objective of the present invention is to provide a fast-acting emergency thermogenic booster that activates controlled thermogenesis to rapidly increase and maintain body temperature, mitigating the effects of hypothermia in survival situations. The present invention consequently provides an effective, portable, and life-saving thermogenic booster for emergency hypothermia prevention. Through multiple delivery methods (i.e., primarily an auto-injector and a capsule), caloric integration, and pharmaceutical-grade safety, the present invention improves cold survival technology.

### BRIEF DESCRIPTION OF THE DRAWINGS

FIG. 1 is a block diagram illustrating a first embodiment of the present invention.

FIG. 2 is a block diagram illustrating a second embodiment of the present invention.

FIG. 3 is a block diagram illustrating an exemplary embodiment of the present invention

FIG. 4 is a block diagram illustrating a first pellet for the exemplary embodiment.

FIG. 5 is a block diagram illustrating a second pellet for the exemplary embodiment.

FIG. 6 is a block diagram illustrating a third pellet for the exemplary embodiment.

### DETAILED DESCRIPTION OF THE INVENTION

All illustrations of the drawings are for the purpose of describing selected versions of the present invention and are not intended to limit the scope of the present invention.

The present invention is a thermogenic composition for cold exposure and is administered in some form to a user in order to prevent the adverse effects of cold exposure and/or to mitigate hypothermia. A first embodiment of the present invention is administered as an injectable formulation to a user, while a second embodiment of the present invention is administered as a capsulated formulation to a user.

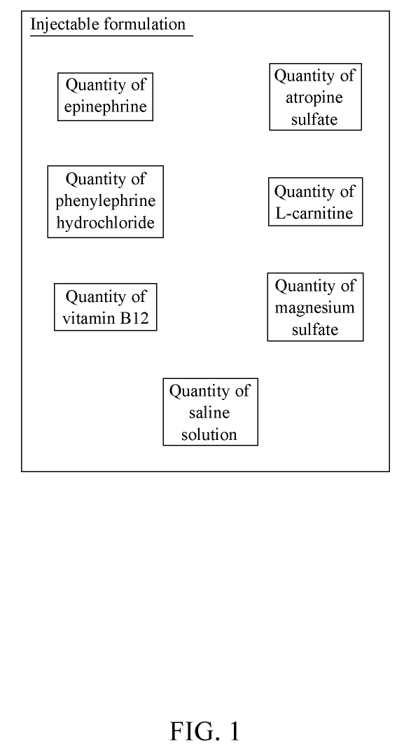

As can be seen in FIG. 1, the first embodiment of the present invention provides a rapid onset emergency injectable solution that immediately raises core body temperature, improves peripheral circulation, and restores metabolic energy during severe hypothermic emergencies. The administration of this embodiment can be carried out by trained first responders, search and rescue teams, military personnel, or medical professionals. Moreover, the first embodiment of the present invention comprises a quantity of epinephrine, a quantity of atropine sulfate, a quantity of phenylephrine hydrochloride, a quantity of L-carnitine, a quantity of vitamin B12, and a quantity of magnesium sulfate, a quantity of saline solution. The quantity of epinephrine (i.e., adrenaline) is used to rapidly elevate core body temperature, to stimulate metabolism, to increase heart rate, and to enhance peripheral circulation. The quantity of atropine sulfate is used to prevent bradycardia (i.e., dangerously slow heart rate) that is common in severe hypothermia and helps maintain adequate cardiac output. The quantity of phenylephrine hydrochloride is used as a potent vasoconstrictor that rapidly restores central body heat and counteracts peripheral heat loss. The quantity of L-carnitine is used to boost mitochondrial energy production, aiding cellular energy levels under hypothermic stress. The quantity of vitamin B12 (i.e., cyanocobalamin) allows for immediate energy metabolism, neurological protection, and rapid circulatory response. The quantity of magnesium sulfate is used to stabilize cardiac rhythm and to prevent arrhythmias triggered by hypothermia. The quantity of saline solution is preferably a buffered isotonic saline solution and allows for isotonic delivery, optimal bioavailability, rapid absorption, and minimal injection discomfort. Thus, the quantity of epinephrine, the quantity of atropine sulfate, the quantity of phenylephrine hydrochloride, the quantity of L-carnitine, the quantity of vitamin B12, the quantity of magnesium sulfate, and the quantity of saline solution are homogeneously mixed to form an injectable formulation. The injectable formulation is preferably administered at an intramuscular location via an auto-injector pen. More specifically, the intramuscular location is at the thigh (i.e., vastus lateralis muscle) with an onset of action within 1 to 3 minutes and a duration of action between 45 to 90 minutes.

The injectable formulation may be constituted with the following proportions in order to rapidly reverse acute hypothermia symptoms, to significantly improve cardiac function and peripheral circulation within minutes, to rapidly stabilize core body temperature, and to immediately enhance metabolic energy. The quantity of epinephrine may approximately be 0.0221 percentage by weight (wt.%) of the injectable formulation, wherein the term “approximately” for the quantity of epinephrine preferably means within an error range of up to ±0.0005 wt.%. The quantity of atropine sulfate may approximately be 0.0368 wt.% of the injectable formulation, wherein the term “approximately” for the quantity of atropine sulfate preferably means within an error range of up to ±0.0005 wt.%. The quantity of phenylephrine hydrochloride may approximately be 0.0737 wt.% of the injectable formulation, wherein the term “approximately” for the quantity of phenylephrine hydrochloride preferably means within an error range of up to ±0.0005 wt.%. The quantity of L-carnitine may approximately be 18.4 wt.% of the injectable formulation, wherein the term “approximately” for the quantity of L-carnitine preferably means within an error range of up to ±0.05 wt.%. The quantity of vitamin B12 may approximately be 0.0737 wt.% of the injectable formulation, wherein the term “approximately” for the quantity of vitamin B12 preferably means within an error range of up to ±0.0005 wt.%. The quantity of magnesium sulfate may approximately be 7.37 wt.% of the injectable formulation, wherein the term “approximately” for the quantity of magnesium sulfate preferably means within an error range of up to ±0.005 wt.%. The quantity of saline solution may approximately be 74.0 wt.% of the injectable formulation, wherein the term “approximately” for the quantity of saline solution preferably means within an error range of up to ±0.05 wt.%.

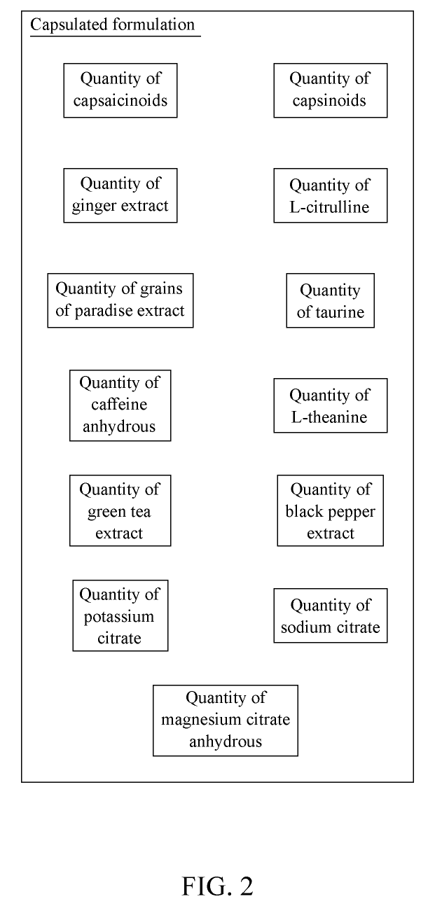

As can be seen in FIG. 2, the second embodiment of the present invention provides the same therapeutic benefits as the first embodiment but at a slower rate. The second embodiment of the invention can also be administered to a user without the supervision of a medical professional or a trained individual. Moreover, the second embodiment of the present invention comprises a quantity of capsaicinoids, a quantity of capsinoids, a quantity of ginger extract, a quantity of L-citrulline, a quantity of grains of paradise extract, a quantity of taurine, a quantity of caffeine anhydrous, a quantity of L-theanine, a quantity of green tea extract, a quantity of black pepper extract, a quantity of potassium citrate, a quantity of sodium citrate, and a quantity of magnesium citrate anhydrous. The quantity of capsaicinoids (i.e., from cayenne) is used to provide a thermogenic effect on a user [i.e., transient receptor potential cation channel subfamily V member 1 (TRPV1)]. The quantity of capsinoids (i.e., CH-19 sweet pepper extract) is also used to provide a thermogenic effect on a user (i.e., non-pungent). The quantity of ginger extract (i.e., ≥5% gingerols) is used to provide warmth and gastrointestinal comfort to a user. The quantity of L-citrulline is used to promote circulation and/or vasodilation for a user. The quantity of grains of paradise extract is used to provide extended warmth to a user. The quantity of taurine is used to provide neurological support and/or mental clarity. The quantity of caffeine anhydrous (i.e., a natural source) is used to provide energy to a user and/or an onset cue to a user. The quantity of L-theanine is used to provide focus smoothing. The quantity of green tea extract [i.e., decaf, ≥50% epigallocatechin gallate (EGCG)] is used in thermogenic signaling for a user. The quantity of black pepper extract (i.e., ≥95% piperine) is used as a bioavailability booster for a user [i.e., drug-drug interaction (DDI)]. The quantity of potassium citrate (i.e., ≈36% K) is used as an electrolyte for a user [i.e., K⁺ ~100 milligrams (mg) per serving]. The quantity of sodium citrate (i.e., ≈ 23.5% N) is also used as an electrolyte for a user (i.e., Na⁺ ~70 mg per serving). The quantity of magnesium citrate anhydrous (i.e., ≈ 15.5% Mg) is also used as an electrolyte for a user (i.e., Mg²⁺ ~20 mg per serving). Thus, the quantity of capsaicinoids, the quantity of capsinoids, the quantity of ginger extract, the quantity of L-citrulline, the quantity of grains of paradise extract, the quantity of taurine, the quantity of caffeine anhydrous, the quantity of L-theanine, the quantity of green tea extract, the quantity of black pepper extract, the quantity of potassium citrate, the quantity of sodium citrate, and the quantity of magnesium citrate anhydrous are heterogeneously mixed to form a capsulated formulation.

The capsulated formulation may be constituted with the following proportions in order to provide a thermogenic activation effect, a circulation support effect, a neurologic-energy modulation effect, and an electrolyte effect. The quantity of capsaicinoids may approximately be 0.267 wt.% of the capsulated formulation, wherein the term “approximately” for the quantity of capsaicinoids preferably means within an error range of up to ±0.0005 wt.%. The quantity of capsinoids may approximately be 0.240 wt.% of the capsulated formulation, wherein the term “approximately” for the quantity of capsinoids preferably means within an error range of up to ±0.0005 wt.%. The quantity of ginger extract may approximately be 2.40 wt.% of the capsulated formulation, wherein the term “approximately” for the quantity of ginger extract preferably means within an error range of up to ±0.005 wt.%. The quantity of L-citrulline may approximately be 28.0 wt.% of the capsulated formulation, wherein the term “approximately” for the quantity of L-citrulline preferably means within an error range of up to ±0.05 wt.%. The quantity of grains of paradise extract may approximately be 0.800 wt.% of the capsulated formulation, wherein the term “approximately” for the quantity of grains of paradise extract preferably means within an error range of up to ±0.0005 wt.%. The quantity of taurine may approximately be 20.0 wt.% of the capsulated formulation, wherein the term “approximately” for the quantity of taurine preferably means within an error range of up to ±0.05 wt.%. The quantity of caffeine anhydrous may approximately be 6.00 wt.% of the capsulated formulation, wherein the term “approximately” for the quantity of caffeine anhydrous preferably means within an error range of up to ±0.005 wt.%. The quantity of L-theanine may approximately be 6.00 wt.% of the capsulated formulation, wherein the term “approximately” for the quantity of L-theanine preferably means within an error range of up to ±0.005 wt.%. The quantity of green tea extract may approximately be 8.00 wt.% of the capsulated formulation, wherein the term “approximately” for the quantity of green tea extract preferably means within an error range of up to ±0.005 wt.%. The quantity of black pepper extract may approximately be 0.120 wt.% of the capsulated formulation, wherein the term “approximately” for the quantity of black pepper extract preferably means within an error range of up to ±0.0005 wt.%. The quantity of potassium citrate may approximately be 11.1 wt.% of the capsulated formulation, wherein the term “approximately” for the quantity of potassium citrate preferably means within an error range of up to ±0.05 wt.%. The quantity of sodium citrate may approximately be 11.9 wt.% of the capsulated formulation, wherein the term “approximately” for the quantity of sodium citrate preferably means within an error range of up to ±0.05 wt.%. The quantity of magnesium citrate anhydrous may approximately be 5.16 wt.% of the capsulated formulation, wherein the term “approximately” for the quantity of magnesium citrate anhydrous preferably means within an error range of up to ±0.005 wt.%.

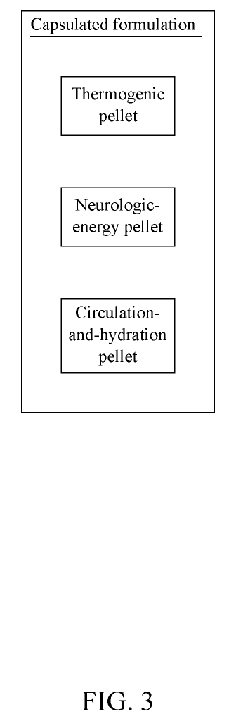

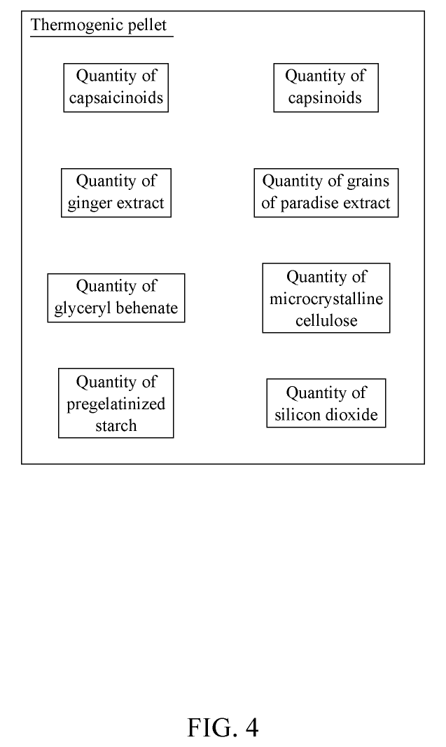

As can be seen in FIG. 3, in an exemplary embodiment of the present invention, the capsulated formulation may be manufactured with three different pellets within a single capsule. As can be seen in FIG. 4, a first pellet within the single capsule is a thermogenic pellet, which includes a quantity of capsaicinoids, a quantity of capsinoids, a quantity of ginger extract (i.e., ≥5% gingerols), a quantity of grains of paradise extract, a quantity of glyceryl behenate (i.e., a lipid carrier), a quantity of microcrystalline cellulose (MCC), a quantity of pregelatinized starch, and a quantity of silicon dioxide. Moreover, the thermogenic pellet may be constituted with the following proportions in order to provide a thermogenic activation effect to a user. The quantity of capsaicinoids may approximately be 3.66 wt.% of the thermogenic pellet, wherein the term “approximately” for the quantity of capsaicinoids preferably means within an error range of up to ±0.005 wt.%. The quantity of capsinoids may approximately be 3.30 wt.% of the thermogenic pellet, wherein the term “approximately” for the quantity of capsinoids preferably means within an error range of up to ±0.005 wt.%. The quantity of ginger extract may approximately be 33.0 wt.% of the thermogenic pellet, wherein the term “approximately” for the quantity of ginger extract preferably means within an error range of up to ±0.05 wt.%. The quantity of grains of paradise extract may approximately be 11.0 wt.% of the thermogenic pellet, wherein the term “approximately” for the quantity of grains of paradise extract preferably means within an error range of up to ±0.05 wt.%. The quantity of glyceryl behenate may approximately be 22.0 wt.% of the thermogenic pellet, wherein the term “approximately” for the quantity of glyceryl behenate preferably means within an error range of up to ±0.05 wt.%. The quantity of MCC may approximately be 14.7 wt.% of the thermogenic pellet, wherein the term “approximately” for the quantity of microcrystalline cellulose preferably means within an error range of up to ±0.05 wt.%. The quantity of pregelatinized starch may approximately be 11.0 wt.% of the thermogenic pellet, wherein the term “approximately” for the quantity of pregelatinized starch preferably means within an error range of up to ±0.05 wt.%. The quantity of silicon dioxide may approximately be 1.47 wt.% of the thermogenic pellet, wherein the term “approximately” for the quantity of silicon dioxide preferably means within an error range of up to ±0.005 wt.%.

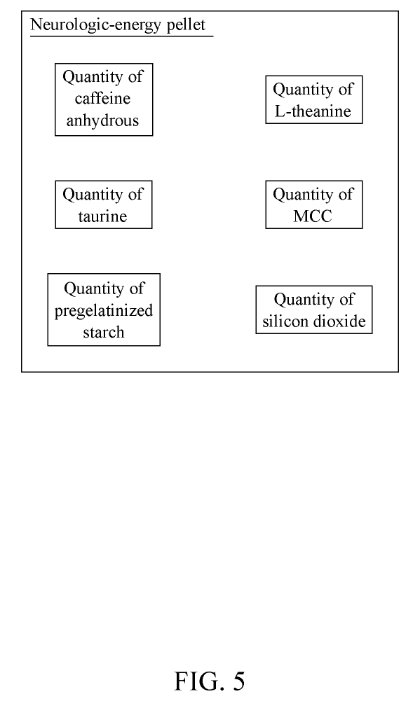

As can be seen in FIG. 5, in this exemplary embodiment of the present invention, a second pellet within the single capsule is a neurologic-energy pellet, which includes a quantity of caffeine anhydrous, a quantity of L-theanine, a quantity of taurine, a quantity of MCC, a quantity of pregelatinized starch, and a quantity of silicon dioxide. Moreover, the neurologic-energy pellet may be constituted with the following proportions in order to provide a neurologic-energy modulation effect to a user. The quantity of caffeine anhydrous may approximately be 16.4 wt.% of the neurologic-energy pellet, wherein the term “approximately” for the quantity of caffeine anhydrous preferably means within an error range of up to ±0.05 wt.%. The quantity of L-theanine may approximately be 16.4 wt.% of the neurologic-energy pellet, wherein the term “approximately” for the quantity of L-theanine preferably means within an error range of up to ±0.05 wt.%. The quantity of taurine may approximately be 54.7 wt.% of the neurologic-energy pellet, wherein the term “approximately” for the quantity of taurine preferably means within an error range of up to ±0.05 wt.%. The quantity of MCC may approximately be 7.66 wt.% of the neurologic-energy pellet, wherein the term “approximately” for the quantity of MCC preferably means within an error range of up to ±0.005 wt.%. The quantity of pregelatinized starch may approximately be 4.38 wt.% of the neurologic-energy pellet, wherein the term “approximately” for the quantity of pregelatinized starch preferably means within an error range of up to ±0.005 wt.%. The quantity of silicon dioxide may approximately be 0.438 wt.% of the neurologic-energy pellet, wherein the term “approximately” for the quantity of silicon dioxide preferably means within an error range of up to ±0.0005 wt.%.

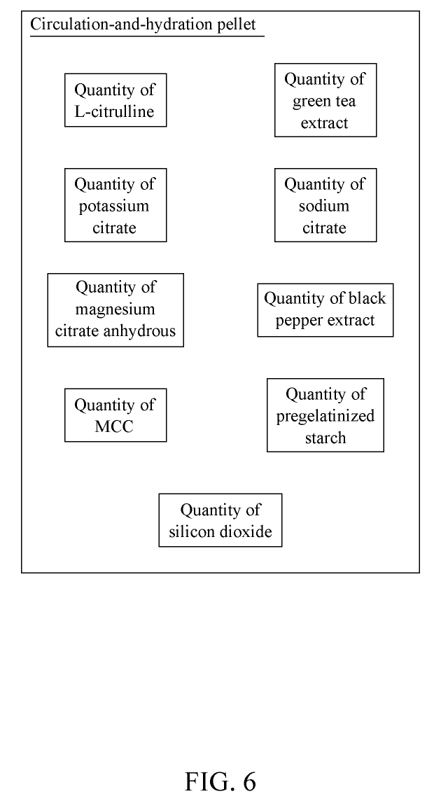

As can be seen in FIG. 6, in this exemplary embodiment of the present invention, a third pellet within the single capsule is a circulation-and-hydration pellet, which includes a quantity of L-citrulline, a quantity of green tea extract, a quantity of potassium citrate, a quantity of sodium citrate, a quantity of magnesium citrate anhydrous, a quantity of black pepper extract, a quantity of MCC, a quantity of pregelatinized starch, and a quantity of silicon dioxide. Moreover, the circulation-and-hydration pellet may be constituted with the following proportions in order to provide a circulation support effect and an electrolyte effect to a user. The quantity of L-citrulline may approximately be 50.0 wt.% of the neurologic-energy pellet, wherein the term “approximately” for the quantity of L-citrulline preferably means within an error range of up to ±0.05 wt.%. The quantity of green tea extract may approximately be 9.51 wt.% of the neurologic-energy pellet, wherein the term “approximately” for the quantity of green tea extract preferably means within an error range of up to ±0.005 wt.%. The quantity of potassium citrate may approximately be 13.2 wt.% of the neurologic-energy pellet, wherein the term “approximately” for the quantity of potassium citrate preferably means within an error range of up to ±0.05 wt.%. The quantity of sodium citrate may approximately be 14.1 wt.% of the neurologic-energy pellet, wherein the term “approximately” for the quantity of sodium citrate preferably means within an error range of up to ±0.05 wt.%. The quantity of magnesium citrate anhydrous may approximately be 6.13 wt.% of the neurologic-energy pellet, wherein the term “approximately” for the quantity of magnesium citrate anhydrous preferably means within an error range of up to ±0.005 wt.%. The quantity of black pepper extract may approximately be 0.142 wt.% of the neurologic-energy pellet, wherein the term “approximately” for the quantity of black pepper extract preferably means within an error range of up to ±0.0005 wt.%. The quantity of MCC may approximately be 4.28 wt.% of the neurologic-energy pellet, wherein the term “approximately” for the quantity of MCC preferably means within an error range of up to ±0.05 wt.%. The quantity of pregelatinized starch may approximately be 2.38 wt.% of the neurologic-energy pellet, wherein the term “approximately” for the quantity of pregelatinized starch preferably means within an error range of up to ±0.005 wt.%. The quantity of silicon dioxide may approximately be 0.285 wt.% of the neurologic-energy pellet, wherein the term “approximately” for the quantity of silicon dioxide preferably means within an error range of up to ±0.0005 wt.%.

Although the invention has been explained in relation to its preferred embodiment, it is to be understood that many other possible modifications and variations can be made without departing from the spirit and scope of the invention as hereinafter claimed.

What is claimed is:

1. A thermogenic composition for cold exposure comprising:
a quantity of epinephrine;
a quantity of atropine sulfate;
a quantity of phenylephrine hydrochloride;
a quantity of L-carnitine;
a quantity of vitamin B12;
a quantity of magnesium sulfate;
a quantity of saline solution; and
the quantity of epinephrine, the quantity of atropine sulfate, the quantity of phenylephrine hydrochloride, the quantity of L-carnitine, the quantity of vitamin B12, the quantity of magnesium sulfate, and the quantity of saline solution being homogeneously mixed to form an injectable formulation.

2. The thermogenic composition for cold exposure as claimed in claim 1, wherein the quantity of epinephrine is approximately 0.0221 percentage by weight (wt.%) of the injectable formulation.

3. The thermogenic composition for cold exposure as claimed in claim 1, wherein the quantity of atropine sulfate is approximately 0.0368 wt.% of the injectable formulation.

4. The thermogenic composition for cold exposure as claimed in claim 1, wherein the quantity of phenylephrine hydrochloride is approximately 0.0737 wt.% of the injectable formulation.

5. The thermogenic composition for cold exposure as claimed in claim 1, wherein the quantity of L-carnitine is approximately 18.4 wt.% of the injectable formulation.

6. The thermogenic composition for cold exposure as claimed in claim 1, wherein the quantity of vitamin B12 is approximately 0.0737 wt.% of the injectable formulation.

7. The thermogenic composition for cold exposure as claimed in claim 1, wherein the quantity of magnesium sulfate is approximately 7.37 wt.% of the injectable formulation.

8. The thermogenic composition for cold exposure as claimed in claim 1, wherein the quantity of saline solution is approximately 74.0 wt.% of the injectable formulation.

9. The thermogenic composition for cold exposure as claimed in claim 1, wherein the quantity of saline solution is a buffered isotonic saline solution.

10. A thermogenic composition for cold exposure comprising:
a quantity of capsaicinoids;
a quantity of capsinoids;
a quantity of ginger extract;
a quantity of L-citrulline;
a quantity of grains of paradise extract;
a quantity of taurine;
a quantity of caffeine anhydrous;
a quantity of L-theanine;
a quantity of green tea extract;
a quantity of black pepper extract;
a quantity of potassium citrate;
a quantity of sodium citrate;
a quantity of magnesium citrate anhydrous; and
the quantity of capsaicinoids, the quantity of capsinoids, the quantity of ginger extract, the quantity of L-citrulline, the quantity of grains of paradise extract, the quantity of taurine, the quantity of caffeine anhydrous, the quantity of L-theanine, the quantity of green tea extract, the quantity of black pepper extract, the quantity of potassium citrate, the quantity of sodium citrate, and the quantity of magnesium citrate anhydrous being heterogeneously mixed to form a capsulated formulation.

11. The thermogenic composition for cold exposure as claimed in claim 10, wherein the quantity of capsaicinoids is approximately 0.267 wt.% of the capsulated formulation.

12. The thermogenic composition for cold exposure as claimed in claim 10, wherein the quantity of capsinoids is approximately 0.240 wt.% of the capsulated formulation.

13. The thermogenic composition for cold exposure as claimed in claim 10, wherein the quantity of ginger extract is approximately 2.40 wt.% of the capsulated formulation.

14. The thermogenic composition for cold exposure as claimed in claim 10, wherein the quantity of L-citrulline is approximately 28.0 wt.% of the capsulated formulation.

15. The thermogenic composition for cold exposure as claimed in claim 10, wherein the quantity of grains of paradise extract is approximately 0.800 wt.% of the capsulated formulation.

16. The thermogenic composition for cold exposure as claimed in claim 10, wherein the quantity of taurine is approximately 20.0 wt.% of the capsulated formulation.

17. The thermogenic composition for cold exposure as claimed in claim 10, wherein the quantity of caffeine anhydrous is approximately 6.00 wt.% of the capsulated formulation.

18. The thermogenic composition for cold exposure as claimed in claim 10, wherein the quantity of L-theanine is approximately 6.00 wt.% of the capsulated formulation.

19. The thermogenic composition for cold exposure as claimed in claim 10, wherein the quantity of green tea extract is approximately 8.00 wt.% of the capsulated formulation.

20. The thermogenic composition for cold exposure as claimed in claim 10, wherein the quantity of black pepper extract is approximately 0.120 wt.% of the capsulated formulation.

21. The thermogenic composition for cold exposure as claimed in claim 10, wherein the quantity of potassium citrate is approximately 11.1 wt.% of the capsulated formulation.

22. The thermogenic composition for cold exposure as claimed in claim 10, wherein the quantity of sodium citrate is approximately 11.9 wt.% of the capsulated formulation.

23. The thermogenic composition for cold exposure as claimed in claim 10, wherein the quantity of magnesium citrate anhydrous is approximately 5.16 wt.% of the capsulated formulation.
